(19) United States
(12) Patent Application Publication
(10) Pub. No.: US 2011/0286988 A1
(43) Pub. Date: Nov. 24, 2011

(54) FVIII Muteins for Treatment of Von Willebrand Disease

(75) Inventors: Haiyan Jiang, San Francisco, CA (US); Glenn Pierce, Rancho Santa Fe, CA (US); John E. Murphy, Berkeley, CA (US); Junliang Pan, Moraga Town, CA (US); Xin Zhang, San Diego, CA (US); Tongyao Liu, Alameda, CA (US)

(73) Assignee: BAYER HEALTHCARE LLC, Tarrytown, NY (US)

(21) Appl. No.: 12/996,629

(22) PCT Filed: Jun. 4, 2009
(86) PCT No.: PCT/US09/46327

§ 371 Date: Jul. 22, 2011

Related U.S. Application Data
(60) Provisional application No. 61/058,795, filed on Jun. 4, 2008.

(51) Int. Cl. A61K 38/48; A61P 7/00; A61P 7/04; C12N 9/96

(52) U.S. Cl. 424/94.3; 435/188

(57) ABSTRACT

This invention relates to treatment of von Willebrand Disease by administration of Factor VIII muteins that are covalently bound, at a predefined site that is not an N-terminal amine, to one or more biocompatible polymers such as polyethylene glycol. The mutein conjugates retain FVIII procoagulant activity and have improved pharmacokinetic properties in subjects lacking von Willebrand Factor.

This application claims benefit of U.S. Provisional Application Ser. No. 61/058,795; filed on Jun. 4, 2008, the contents of which are incorporated herein by reference in their entirety.

### FIELD OF THE INVENTION

This invention relates to Factor VIII (FVIII) muteins, and derivatives thereof, useful for treatment of von Willebrand Disease (vWD). The FVIII muteins allow coupling, at a defined site, to one or more biocompatible polymers such as polyethylene glycol. In addition, related formulations, dosages, and methods of administration thereof for therapeutic purposes are provided. These modified FVIII variants, and associated compositions and methods are useful in providing a treatment option with reduced injection frequency and reduced immunogenic response for individuals afflicted with von Willebrand Disease.

### BACKGROUND OF THE INVENTION

vWD is a term that describes a cluster of hereditary or acquired diseases of various etiologies. The basis of many types of vWD resides in the function of von Willebrand Factor (vWF), which is a series of multimeric plasma glycoproteins that, among other properties, binds to the procoagulant FVIII and extends the half-life of native FVIII in the blood circulation (see, e.g., Federici, Haemophilia 10 (suppl 4):169, 2004; Denis, et al., Thromb. Haemost. 99:271, 2008). In normal people, the half-life of FVIII is approximately 8 minutes in the absence of vWF and 8 hours in the presence of vWF.

In a mild form (Type 1), vWD is very common, affecting as many as one in 100 persons in the population, and affecting men and women equally.

Type 2 vWD can be a severe form of vWD and is known in five subtypes: 2A, 2B, 2C, 2M and 2N. Of these, type 2N is characterized by a deficiency of binding of FVIII to vWF. Thus, in patients with type 2N vWD, FVIII is rapidly degraded and levels in circulation are low. The vWF type 2N is caused by homozygous or compound heterozygous vWF mutations that impair binding to FVIII. Since free FVIII that is not in a complex with vWF is rapidly cleared from the circulation, vWD 2N masquerades as an autosomal recessive form of hemophilia A. However, patients typically have normal levels of vWF-Antigen and Ristocetin cofactor activity for vWF-platelet GPlb binding (vWF:RCo activity), but reduced FVIII levels.

Type 3 vWD, the form Eric von Willebrand originally described in a Finnish family, is a homozygous deficiency of vWF or a double heterozygous deficiency. vWD type 3 is caused by nonsense mutations or frameshifts due to small insertions or deletions into the nucleic acid encoding vWF, which results in a complete or nearly complete deficiency of vWF. In most cases, vWF:RCo and vWF:Ag are undetectable and FVIII levels are profoundly reduced. Patients with Type 3 vWD can have hemarthroses and bleeding into joints or spaces, much like hemophilia.

Acquired vWD is usually caused by autoimmune clearance due to development of anti-vWF antibodies, fluid shear stress-induced proteolysis or increased binding to platelets or other cells. The acquired vWD syndrome is similar to those of vWD type 3, with decreased levels of vWF-Ag, vWF:Rco and FVIII. vWD type 3 and acquired vWD patients not only suffer from mucosal bleeding which is characteristic of vWD but also soft tissue, muscle, and joint bleeding, which are characteristic of hemophilia A.

Hemophilia A is the most common hereditary coagulation disorder, with an estimated incidence of 1 per 5000 males. It is caused by deficiency or structural defects in FVIII, a critical component of the intrinsic pathway of blood coagulation. The current treatment for hemophilia A involves intravenous injection of human FVIII. Human FVIII has been produced recombinantly as a single-chain molecule of approximately 300 kD. It consists of the structural domains A1-A2-B-A3-C1-C2 (Thompson, Semin. Hematol. 29:11-22, 2003). The precursor product is processed into two polypeptide chains of 200 kD (heavy) and 80 kD (light) in the Golgi Apparatus, with the two chains held together by metal ions (Kaufman, et al., J. Biol. Chem. 263:6352, 1988; Andersson, et al., Proc. Natl. Acad. Sci. 83:2979, 1986).

The B-domain of FVIII seems to be dispensable as B-domain deleted FVIII (BDD, 90 kD A1-A2 heavy chain plus 80 kD light chain) has also been shown to be effective as a replacement therapy for hemophilia A. The B-domain deleted FVIII sequence contains a deletion of all but 14 amino acids of the B-domain.

Hemophilia A patients are currently treated by intravenous administration of FVIII on demand or as a prophylactic therapy administered several times a week. For prophylactic treatment 15-25 IU/kg bodyweight is given of FVIII three times a week. It is constantly required in the patient. Because of its short half-life in man, FVIII must be administered frequently. Despite its large size of greater than 300 kD for the full-length protein, FVIII has a half-life in humans of only about 11 hours (Ewenstein, et al., Semin. Hematol. 41:1-16, 2004). The need for frequent intravenous injection creates tremendous barriers to patient compliance. It would be more convenient for the patients if a FVIII product could be developed that had a longer half-life and therefore required less frequent administration. Furthermore, the cost of treatment could be reduced if the half-life were increased because fewer dosages may then be required.

An additional disadvantage to the current therapy is that about 25-30% of patients develop antibodies that inhibit FVIII activity (Saenko, et al., Haemophilia 8:111, 2002). The major epitopes of inhibitory antibodies are located within the A2 domain at residues 484-508, the A3 domain at residues 1811-1818, and the C2 domain. Antibody development prevents the use of FVIII as a replacement therapy, forcing this group of patients to seek an even more expensive treatment with high-dose recombinant Factor VIIa and immune tolerance therapy.

The following studies identified FVIII epitopes of inhibitory antibodies. In a study of 25 inhibitory plasma samples, 11 were found to bind to the thrombin generated 73 kD light chain fragment A3C1C2, 4 to the A2 domain, and 10 to both (Fulcher, et al., Proc. Natl. Acad. Sci. 2:7728-32, 1985). In another study, six of eight A2 domain inhibitors from patients were neutralized by a recombinant A2 polypeptide (Scandella, et al., Blood 82:1767-75, 1993). Epitopes for six of nine inhibitors from patients were mapped to A2 residues 379538 (Scandella, et al., Proc. Natl. Acad. Sci. 85:6152-6, 1988). An epitope for 18 heavy-chain inhibitors was localized to the same N-terminal 18.3 kD region of the A2 domain (Scandella, et al., Blood 74:1618-26, 1989).

An active, recombinant hybrid human/porcine FVIII molecule, generated by replacing human A2 domain residues 387-604 with the homologous porcine sequence, was resistant to a patient A2 inhibitor (Lubin, et al., J. Biol. Chem. 269:8639-41, 1994) and resistant to a murine monoclonal antibody mAB 413 IgG that competes with patient A2 inhibitors for binding to A2 (Scandella, et al., Thromb Haemost. 67:665-71, 1992). This A2 domain epitope was further localized to the A2 domain residues 484-508 when experiments showed that mAB 413 IgG and four patient inhibitors did not inhibit a hybrid human/porcine FVIII in which the A2 domain residues 484-508 were replaced with that of porcine (Healey, et al., J. Biol. Chem. 270:14505-14509, 1995). This hybrid FVIII was also more resistant to at least half of 23 patient plasmas screened (Barrow, et al., Blood 95:564-568, 2000). Alanine scanning mutagenesis identified residue 487 to be critical for binding to all five patient inhibitors tested, while residues 484, 487, 489, and 492 were all important to interaction with mAB 413 IgG (Lubin, J. Biol. Chem. 272:30191-30195, 1997). Inhibitory antibody titers in mice receiving the R484A/R489A/P492A mutant, but not the R484A/R489A mutant, were significantly lower than in mice receiving control human BDD FVIII (Parker, et al., Blood 104:704-710, 2004). In sum, the 484-508 region of the A2 domain seems to be a binding site for inhibitors of FVIII activity.

In addition to the development of an immune response to FVIII, another problem with conventional therapy is that it requires frequent dosaging because of the short half-life of FVIII in vivo. The mechanisms for clearance of FVIII from the circulation have been studied.

FVIII clearance from circulation has been partly attributed to specific binding to the low-density lipoprotein receptor-related protein (LRP), a hepatic clearance receptor with broad ligand specificity (Oldenburg, et al., Haemophilia 10 Suppl 4:133-139, 2004). Recently, the low-density lipoprotein (LDL) receptor was also shown to play a role in FVIII clearance, such as by cooperating with LRP in regulating plasma levels of FVIII (Bovenschen, et al., Blood 106:906-910, 2005). Both interactions are facilitated by binding to cell-surface heparin sulphate proteoglycans (HSPGs). Plasma half-life in mice can be prolonged by 3.3-fold when LRP is blocked or 5.5-fold when both LRP and cell-surface HSPGs are blocked (Sarafanov, et al., J. Biol. Chem. 276:11970-11979, 2001). HSPGs are hypothesized to concentrate FVIII on the cell surface and to present it to LRP. LRP binding sites on FVIII have been localized to A2 residues 484-509 (Saenko, et al., J. Biol. Chem. 274:37685-37692, 1999), A3 residues 1811-1818 (Bovenschen, et al., J. Biol. Chem. 278:9370-9377, 2003), and an epitope in the C2 domain (Lenting, et al., J. Biol. Chem. 274:23734-23739, 1999).

FVIII is also cleared from circulation by the action of proteases. To understand this effect, one must understand the mechanism by which FVIII is involved in blood coagulation. FVIII circulates as a heterodimer of heavy and light chains, bound to vWF. vWF binding involves FVIII residues 1649-1689 (Foster, et al., J. Biol. Chem. 263:5230-5234, 1998), and parts of C1 (Jacquemin, et al., Blood 96:958-965, 2000) and C2 domains (Spiegel, et al., J. Biol. Chem. 279:53691-53698, 2004). FVIII is activated by thrombin, which cleaves peptide bonds after residues 372, 740, and 1689 to generate a heterotrimer of A1, A2, and A3-C1-C2 domains (Pittman, et al., Proc. Natl. Acad. Sci. 276:12434-12439, 2001). Upon activation, FVIII dissociates from vWF and is concentrated to the cell surface of platelets by binding to phospholipid. Phospholipid binding involves FVIII residues 2199, 2200, 2251, and 2252 (Gilbert et al., J. Biol. Chem. 277:6374-6381, 2002). There it binds to FIX through interactions with FVIII residues 558-565 (Fay, et al., J. Biol. Chem. 269:20522-20527, 1994) and 1811-1818 (Lenting, et al., J. Biol. Chem. 271:1935-1940, 1996) and FX through interactions with FVIII residues 349-372 (Nogami, et al., J. Biol. Chem. 279:15763-15771, 2004) and acts as a cofactor for FIX activation of FX, an essential component of the intrinsic coagulation pathway. Activated FVIII (FVIIIa) is partly inactivated by the protease activated protein C (APC) through cleavage after FVIII residues 336 and 562 (Regan, et al., J. Biol. Chem. 271:3982-3987, 1996). The predominant determinant of inactivation, however, is the dissociation of the A2 domain from A1 and A3-C1-C2 (Fay, et al., J. Biol. Chem. 266:8957-8962, 1991).

One method that has been demonstrated to increase the in vivo half-life of a protein is PEGylation. PEGylation is the covalent attachment of long-chained polyethylene glycol (PEG) molecules to a protein or other molecule. The PEG can be in a linear form or in branched form to produce different molecules with different features. Besides increasing the half-life of peptides or proteins, PEGylation has been used to reduce antibody development, protect the protein from protease digestion and keep the material out of the kidney filtrate (Harris, et al., Clinical Pharmacokinetics 40:539-551, 2001). In addition, PEGylation may also increase the overall stability and solubility of the protein. Finally, the sustained plasma concentration of PEGylated proteins can reduce the extent of adverse side effects by reducing the trough to peak levels of a drug, thus eliminating the need to introduce super-physiological levels of protein at early time-points.

Random modification of FVIII by targeting primary amines (N-terminus and lysines) with large polymers such as PEG and dextran has been attempted with varying degree of success (WO94/15625, U.S. Pat. No. 4,970,300, U.S. Pat. No. 6,048,720). The most dramatic improvement, published in a 1994 patent application (WO94/15625), shows a 4-fold half-life improvement but at a cost of 2-fold activity loss after reacting full-length FVIII with 50-fold molar excess of PEG. WO2004/075923 discloses conjugates of FVIII and polyethylene glycol that are created through random modification. Randomly PEGylated proteins, such as interferon-alpha (Kozlowski, et al, BioDrugs 15:419-429, 2001) have been approved as therapeutics in the past.

This random approach, however, is much more problematic for the heterodimeric FVIII. FVIII has hundreds of potential PEGylation sites, including the 158 lysines, the two N-termini, and multiple histidines, serines, threonines, and tyrosines, all of which could potentially be PEGylated with reagents primarily targeting primary amines. For example, the major positional isomer for PEGylated interferon Alpha-2b was shown to be a histidine (Wang, et al., Biochemistry 39:10634-10640, 2000). Furthermore, heterogeneous processing of full length FVIII can lead to a mixture of starting material that leads to further complexity in the PEGylated products. An additional drawback to not controlling the site of PEGylation on FVIII is a potential activity reduction if the PEG were to be attached at or near critical active sites, especially if more than one PEG or a single large PEG is conjugated to FVIII. Because random PEGylation will invariably produce large amounts of multiply PEGylated products, purification to obtain only mono-PEGylated products will drastically lower overall yield. Finally, the enormous heterogeneity in product profile will make consistent synthesis and characterization of each lot nearly impossible. Since good manufacturing requires a consistent, well-characterized product, product heterogeneity is a barrier to commercialization. For all these reasons, a more specific method for PEGylating FVIII is desired.

Various site-directed protein PEGylation strategies have been summarized in a recent review (Kochendoerfer, et al., Curr. Opin. Chem. Biol. 9:555-560, 2005). One approach involves incorporation of an unnatural amino acid into proteins by chemical synthesis or recombinant expression followed by the addition of a PEG derivative that will react specifically with the unnatural amino acid. For example, the unnatural amino acid may be one that contains a keto group not found in native proteins. However, chemical synthesis of proteins is not feasible for a protein as large as FVIII. Current limit of peptide synthesis is about 50 residues. Several peptides can be ligated to form a larger piece of polypeptide, but to produce even the B-domain deleted FVIII would require greater than 20 ligations, which would result in less than 1% recovery even under ideal reaction condition. Recombinant expression of proteins with unnatural amino acids has so far mainly been limited to non-mammalian expression systems. This approach is expected to be problematic for a large and complex protein such as FVIII that needs to be expressed in mammalian systems.

Another approach to site-specific PEGylation of proteins is by targeting N-terminal backbone amines with PEG-aldehydes. The low pH required under this process to achieve specificity over other amine groups, however, is not compatible with the narrow near-neutral pH range needed for the stability of FVIII (Wang, et al., Intl. J. Pharmaceutics 259, pp. 1-15, 2003). Moreover, N-terminal PEGylation of FVIII may not lead to improved plasma half-life if this region is not involved in plasma clearance.

WO90/12874 discloses site-specific modification of human IL-3, granulocyte colony stimulating factor and erythropoietin polypeptides by inserting or substituting a cysteine for another amino acid, then adding a ligand that has a sulfhydryl reactive group. The ligand couples selectively to cysteine residues. Modification of FVIII or any variant thereof is not disclosed.

EP 0 319 315 discloses FVIII muteins having deletions or alterations of the vWF binding site which result in decreased vWF binding. EP 0 319 315 further discloses relief of FVIII deficiency resulting from vWF inhibitory activity by administering such muteins.

Rottensteiner et al. discloses random chemical modification of lysine residues in FVIII to form conjugates with polyethylene glycol or polysialic acid. Blood 110(11), 3150A (2007). Rottensteiner et al. further suggests that randomly modified FVIII may be useful in vWD type 2N.

For the reasons stated above, there exists a need for an improved FVIII variant that possesses greater duration of action in vivo and reduced immunogenicity, while retaining functional activity. Furthermore, it is desirable that such a protein be produced as a homogeneous product in a consistent manner.

### SUMMARY OF THE INVENTION

It is an object of the present invention to provide a method of treating vWD comprising administration of a biocompatible polymer-conjugated functional FVIII polypeptide having improved pharmacokinetic characteristics and therapeutic characteristics.

It is also an object of the present invention to provide a method for treating vWD comprising administering to a subject in need thereof a therapeutically effective amount of a conjugate that has FVIII procoagulant activity and that is capable of correcting human FVIII deficiencies, the conjugate comprising a functional FVIII polypeptide covalently attached at one or more predefined sites on the polypeptide to one or more biocompatible polymers, wherein the predefined site is a particular amino acid residue identified by numerical position in the amino acid sequence of the polypeptide and is not an N-terminal amine. The von Willebrand Disease can be characterized by a deficiency and/or abnormality of von Willebrand Factor.

It is another object of the invention to provide a method of preparing a medicament for treating vWD, comprising making a conjugate that has FVIII procoagulant activity and that is capable of correcting human FVIII deficiencies, the conjugate comprising a functional FVIII polypeptide covalently attached at one or more predefined sites on the polypeptide to one or more biocompatible polymers, wherein the predefined site is a particular amino acid residue identified by numerical position in the amino acid sequence of the polypeptide and is not an N-terminal amine.

It is yet another method of the invention to provide a method for treating vWD, comprising administering to a subject in need thereof a therapeutically effective amount of a cysteine substituted variant of FVIII having FVIII procoagulant activity and capable of correcting human FVIII deficiencies, the variant characterized by having a cysteine residue substituted for an amino acid in the FVIII sequence, wherein said substitution causes a cysteine residue at an amino acid position where a cysteine residue is not present in FVIII with reference to the mature, full-length human FVIII amino acid sequence of SEQ ID NO:1, said cysteine added variant being further characterized by having a biocompatible polymer covalently attached to said substitute cysteine residue.

It is another object of the present invention to provide a method for treating vWD, comprising administration to a subject in need thereof a biocompatible polymer-conjugated B domain deleted FVIII protein having improved pharmacokinetic properties.

It is yet another object of the invention to provide a method for treating vWD, comprising administering to a subject in need thereof a biocompatible polymer-conjugated functional FVIII polypeptide having reduced binding to the low-density lipoprotein receptor-related protein (LRP), low-density lipoprotein (LDL) receptor, the heparan sulphate proteoglycans (HSPGs) and/or inhibitory antibodies against FVIII.

It is yet another object of the present invention to provide a method for treating vWD comprising administration to a subject in need thereof of a therapeutically effective amount of an improved FVIII variant that possesses greater duration of action in vivo and reduced immunogenicity, which is capable of being produced as a homogeneous product in a consistent manner.

In one aspect of the invention there is provided a method for treating vWD comprising administering to a subject in need thereof a therapeutically effective amount of a conjugate having FVIII procoagulant activity comprising a functional FVIII polypeptide covalently attached at one or more predefined sites on the polypeptide to one or more biocompatible polymers, wherein the predefined site is a not an N-terminal amine.

In another aspect of the invention there is provided a method for prophylactic treatment prior to surgery, comprising administering to a subj ect prior to surgery a therapeutically effective amount of a conjugate that has FVIII procoagulant activity and that is capable of correcting human FVIII deficiencies, the conjugate comprising a functional FVIII polypeptide covalently attached at one or more predefined sites on the polypeptide to one or more biocompatible polymers, wherein the predefined site is a particular amino acid residue identified by numerical position in the amino acid sequence of the polypeptide and is not an N-terminal amine, whereby episodic bleeding is attenuated. The subject can have vWD, for example Type 3 vWD. Advantageously, the conjugate is administered within 24 hours before surgery, preferably within eight hours, most preferably from 0.5 to two hours before surgery.

In yet another aspect of the invention, there is provided a method for treatment of trauma comprising administering to in a subject in need thereof a therapeutically effective amount of a conjugate that has FVIII procoagulant activity and that is capable of correcting human FVIII deficiencies, the conjugate comprising a functional FVIII polypeptide covalently attached at one or more predefined sites on the polypeptide to one or more biocompatible polymers, wherein the predefined site is a particular amino acid residue identified by numerical position in the amino acid sequence of the polypeptide and is not an N-terminal amine, whereby episodic bleeding is attenuated. The subject can have vWD, including Type 3 vWD.

### BRIEF DESCRIPTION OF THE FIGURES

FIG. 1. Effect of PEGylated FVIII to restore FVIII half-life to normal in vWD Knock-out (KO) mice. The FIGURE illustrates the time course of plasma FVIII activity upon i) administration of rFVIII to vWF KO mice (filled circles), ii) administration of rFVIII to FVIII KO mice (open circles), iii) administration of a PEGylated rFVIII to vWF KO mice (64 kD PEG14, filled squares), and iv) administration of a differently PEGylated rFVIII to vWF KO mice (64 kD PEG2+14, filled triangles).

### DETAILED DESCRIPTION OF THE INVENTION

The present invention is based on the discovery that that polypeptides having FVIII activity can be covalently attached at a predefined site to a biocompatible polymer that is not at an N-terminal amine, and that such polypeptides substantially retain their coagulant activity. Furthermore, these polypeptide conjugates have improved circulation time and reduced antigenicity.

The present invention is further based on the discovery that FVIII muteins covalently linked to a biocompatible polymer at a predefined site have a longer half-life of procoagulant activity in the circulation of subjects lacking vWF than does unmodified FVIII. Treatment of a subject substantially lacking vWF using the conjugates of the invention can be advantageous over using prior art conjugates that have random polymer attachments to FVIII or attachments at an N-terminal. Site-directed attachment allows one to design modifications that avoid the regions required for biological activity and thereby to maintain substantial FVIII activity. It also allows for designing to attach polymers to block binding at sites involved in FVIII clearance. Site-directed attachment also allows for a uniform product rather than the heterogeneous conjugates produced in the art by random polymer coupling. By avoiding attachment at an N-terminal amine of the light chain, the conjugates of the present invention avoid the possible loss of activity from attaching a ligand at an active site of the FVIII polypeptide.

### DEFINITIONS

Biocompatible polymer. A biocompatible polymer includes polyalkylene oxides such as without limitation polyethylene glycol (PEG), dextrans, colominic acids or other carbohydrate based polymers, polymers of amino acids, biotin derivatives, polyvinyl alcohol (PVA), polycarboxylates, polyvinylpyrrolidone, polyethylene-co-maleic acid anhydride, polystyrene-co-malic acid anhydride, polyoxazoline, polyacryloylmorpholine, heparin, albumin, celluloses, hydrolysates of chitosan, starches such as hydroxyethyl-starches and hydroxy propyl-starches, glycogen, agaroses and derivatives thereof, guar gum, pullulan, inulin, xanthan gum, carrageenan, pectin, alginic acid hydrolysates, other bio-polymers and any equivalents thereof. An example of a polymer is a polyethylene glycol such as methoxypolyethylene glycol (mPEG). Other useful polyalkylene glycol compounds are polypropylene glycols (PPG), polybutylene glycols (PBG), PEG-glycidyl ethers (Epox-PEG), PEG-oxycarbonylimidazole (CDI-PEG), branched polyethylene glycols, linear polyethylene glycols, forked polyethylene glycols and multiarmed or “super branched” polyethylene glycols (star-PEG).

Polyethylene glycol (PEG). “PEG” and “polyethylene glycol” as used herein are interchangeable and include any water-soluble poly(ethylene oxide). Typically, PEGs for use in accordance with the invention comprise the following structure “—(OCH2CH2)n-” where (n) is 2 to 4000. As used herein, PEG also includes “—CH2CH2-O(CH2CH2O)n-CH2CH2-” and “—(OCH2CH2)nO—,” depending upon whether or not the terminal oxygens have been displaced.

Throughout the specification and claims, it should be remembered that the term “PEG” includes structures having various terminal or “end capping” groups, such as without limitation a hydroxyl or a C1-20 alkoxy group. The term “PEG” also means a polymer that contains a majority, that is to say, greater than 50%, of —OCH2CH2-repeating subunits. With respect to specific forms, the PEG can take any number of a variety of molecular weights, as well as structures or geometries such as branched, linear, forked, and multifunctional.

PEGylation. PEGylation is a process whereby a polyethylene glycol (PEG) is covalently attached to a molecule such as a protein.

Activated or active functional group. When a functional group such as a biocompatible polymer is described as activated, the functional group reacts readily with an electrophile or a nucleophile on another molecule.

B domain deleted FVIII (BDD). As used herein, BDD is characterized by having the amino acid sequence which contains a deletion of all but 14 amino acids of the B-domain of FVIII. The first 4 amino acids of the B-domain (SFSQ, SEQ ID NO:2) are linked to the 10 last residues of the B-domain (NPPVLKRHQR, SEQ ID NO:3) (Lind, et al, Eur. J. Biochem. 232:19-27, 1995). The BDD used herein has the amino acid sequence of SEQ ID NO:4. Examples of BDD polypeptides are described in WO 2006/053299 which is incorporated herein by reference.

FVIII. Blood clotting Factor VIII (FVIII) is a glycoprotein synthesized and released into the bloodstream by the liver. In the circulating blood, it is bound to von Willebrand factor (vWF, also known as FVIII-related antigen) to form a stable complex. Upon activation by thrombin, it dissociates from the complex to interact with other clotting factors in the coagulation cascade, which eventually leads to the formation of a thrombus. Human full-length FVIII has the amino acid sequence of SEQ ID NO:1, although allelic variants are possible.

Functional FVIII polypeptide. As used herein, functional FVIII polypeptide denotes a functional polypeptide or combination of polypeptides that are capable, in vivo or in vitro, of correcting human FVIII deficiencies, characterized, for example, by hemophilia A. FVIII has multiple degradation or processed forms in the natural state. These are proteolytically derived from a precursor, one chain protein, as demonstrated herein. A functional FVIII polypeptide includes such single chain protein and also provides for these various degradation products that have the biological activity of correcting human FVIII deficiencies. Allelic variations likely exist. The functional FVIII polypeptides include all such allelic variations, glycosylated versions, modifications and fragments resulting in derivatives of FVIII so long as they contain the functional segment of human FVIII and the essential, characteristic human FVIII functional activity remains unaffected in kind. Those derivatives of FVIII possessing the requisite functional activity can readily be identified by straightforward in vitro tests described herein. Furthermore, functional FVIII polypeptide is capable of catalyzing the conversion of Factor X (FX) to FXa in the presence of FIXa, calcium, and phospholipid, as well as correcting the coagulation defect in plasma derived from hemophilia A affected individuals. From the disclosure of the sequence of the human FVIII amino acid sequences and the functional regions herein, the fragments that can be derived via restriction enzyme cutting of the DNA or proteolytic or other degradation of human FVIII protein will be apparent to those skilled in the art. Examples of functional FVIII polypeptides are described in WO 2006/053299 which is incorporated herein by reference.

FIX. As used herein, FIX means Coagulation Factor IX, which is also known as Human Clotting Factor IX, or Plasma Thromboplastin Component.

FX. As used herein, FX means Coagulation Factor X, which is also known by the names Human Clotting Factor X and by the eponym Stuart-Prower factor.

Pharmacokinetics. “Pharmacokinetics” (“PK”) is a term used to describe the properties of absorption, distribution, metabolism, and elimination of a drug in a body. An improvement to a drug's pharmacokinetics means an improvement in those characteristics that make the drug more effective in vivo as a therapeutic agent, especially its useful duration in the body.

Mutein. A mutein is a genetically engineered protein arising as a result of a laboratory induced mutation to a protein or polypeptide.

Protein. As used herein, protein and polypeptide are synonyms.

FVIII clearance receptor. A FVIII clearance receptor as used herein means a receptor region on a functional FVIII polypeptide that binds or associates with one or more other molecules to result in FVIII clearance from the circulation. FVIII clearance receptors include without limitation the regions of the FVIII molecule that bind LRP, LDL receptor and/or HSPG.

It is envisioned that any functional FVIII polypeptide may be mutated at a predetermined site and then covalently attached at that site to a biocompatible polymer according to the methods of the invention. Useful polypeptides include, without limitation, full-length FVIII having the amino acid sequence as shown in SEQ ID NO:1 and BDD FVIII having the amino acid sequence as shown in SEQ ID NO:4.

The biocompatible polymer used in the conjugates of the invention may be any of the polymers discussed above. The biocompatible polymer is selected to provide the desired improvement in pharmacokinetics. For example, the identity, size and structure of the polymer is selected so as to improve the circulation half-life of the polypeptide having FVIII activity or decrease the antigenicity of the polypeptide without an unacceptable decrease in activity. The polymer may comprise PEG, and as an example, may have at least 50% of its molecular weight as PEG. In one embodiment, the polymer is a polyethylene glycol terminally capped with an end-capping moiety such as hydroxyl, alkoxy, substituted alkoxy, alkenoxy, substituted alkenoxy, alkynoxy, substituted alkynoxy, aryloxy and substituted aryloxy. In another embodiment, the polymers may comprise methoxypolyethylene glycol. In a further embodiment, the polymers may comprise methoxypolyethylene glycol having a size range from 3 kD to 100 kD, or from 5 kD to 64 kD, or from 5 kD to 43 kD.

The polymer may have a reactive moiety. For example, in one embodiment, the polymer has a sulfhydryl reactive moiety that can react with a free cysteine on a functional FVIII polypeptide to form a covalent linkage. Such sulfhydryl reactive moieties include thiol, triflate, tresylate, aziridine, oxirane, 5-pyridyl, or maleimide moieties. In one embodiment, the polymer is linear and has a “cap” at one terminus that is not strongly reactive towards sulfhydryls (such as methoxy) and a sulfhydryl reactive moiety at the other terminus. In one embodiment, the conjugate comprises PEG-maleimide and has a size range from 5 kD to 64 kD.

Further guidance for selecting useful biocompatible polymers is provided in the examples that follow.

Site-directed mutation of a nucleotide sequence encoding polypeptide having FVIII activity may occur by any method known in the art. Methods include mutagenesis to introduce a cysteine codon at the site chosen for covalent attachment of the polymer. This may be accomplished using a commercially available site-directed mutagenesis kit such as the Stratagene cQuickChange™ II site-directed mutagenesis kit, the Clontech Transformer site-directed mutagenesis kit no. K1600-1, the Invitrogen GenTaylor site-directed mutagenesis system no. 12397014, the Promega Altered Sites II in vitro mutagenesis system kit no. Q6210, or the Takara Mirus Bio LA PCR mutagenesis kit no. TAK RR016.

The conjugates of the invention may be prepared by first replacing the codon for one or more amino acids on the surface of the functional FVIII polypeptide with a codon for cysteine, producing the cysteine mutein in a recombinant expression system, reacting the mutein with a cysteine-specific polymer reagent, and purifying the mutein.

In this system, the addition of a polymer at the cysteine site can be accomplished through a maleimide active functionality on the polymer. Examples of this technology are provided infra. The amount of sulfhydryl reactive polymer used should be at least equimolar to the molar amount of cysteines to be derivatized and preferably is present in excess. As an example, at least a 5-fold molar excess of sulfhydryl reactive polymer is used, or at least a ten-fold excess of such polymer is used. Other conditions useful for covalent attachment are within the skill of those in the art.

In the examples that follow, the muteins are named in a manner conventional in the art. The convention for naming mutants is based on the amino acid sequence for the mature, full length FVIII as provided in SEQ ID NO:1. As a secreted protein, FVIII contains a signal sequence that is proteolytically cleaved during the translation process. Following removal of the 19 amino acid signal sequence, the first amino acid of the secreted FVIII product is an alanine.

As is conventional and used herein, when referring to mutated amino acids in BDD FVIII, the mutated amino acid is designated by its position in the sequence of full-length FVIII. For example, the PEG6 mutein discussed below is designated K1808C because it changes the lysine (K) at the position analogous to 1808 in the full-length sequence to cysteine (C).

The predefined site for covalent binding of the polymer is best selected from sites exposed on the surface of the polypeptide that are not involved in FVIII activity. Such sites are also best selected from those sites known to be involved in mechanisms by which FVIII is deactivated or cleared from circulation. Selection of these sites is discussed in detail below. Preferred sites include an amino acid residue in or near a binding site for (a) low density lipoprotein receptor related protein, (b) a heparin sulphate proteoglycan, (c) low density lipoprotein receptor, and/or (d) FVIII inhibitory antibodies. By “in or near a binding site” means a residue that is sufficiently close to a binding site such that covalent attachment of a biocompatible polymer to the site would result in steric hindrance of the binding site. Such a site is expected to be within 20 Å of a binding site, for example.

In one embodiment of the invention, the biocompatible polymer is covalently attached to the functional FVIII polypeptide at an amino acid residue in or near (a) a binding site for a protease capable of degradation of FVIII and/or (b) a binding site for FVIII inhibitory antibodies. The protease may be activated protein C (APC). In another embodiment, the biocompatible polymer is covalently attached at the predefined site on the functional FVIII polypeptide such that binding of low-density lipoprotein receptor related protein to the polypeptide is less than to the polypeptide when it is not conjugated, for example, more than twofold less. In one embodiment, the biocompatible polymer is covalently attached at the predefined site on the functional FVIII polypeptide such that binding of heparin sulphate proteoglycans to the polypeptide is less than to the polypeptide when it is not conjugated, for example, more than twofold less. In a further embodiment, the biocompatible polymer is covalently attached at the predefined site on the functional FVIII polypeptide such that binding of FVIII inhibitory antibodies to the polypeptide is less than to the polypeptide when it is not conjugated, for example, more than twofold less than the binding to the polypeptide when it is not conjugated. In another embodiment, the biocompatible polymer is covalently attached at the predefined site on the functional FVIII polypeptide such that binding of low density lipoprotein receptor to the polypeptide is less than to the polypeptide when it is not conjugated, for example, more than twofold less. In another embodiment, the biocompatible polymer is covalently attached at the predefined site on the functional FVIII polypeptide such that a plasma protease degrades the polypeptide less than when the polypeptide is not conjugated. In a further embodiment, the degradation of the polypeptide by the plasma protease is more than twofold less than the degradation of the polypeptide when it is not conjugated as measured under the same conditions over the same time period.

LRP, LDL receptor, or HSPG binding affinity for FVIII can be determined using surface plasmon resonance technology (Biacore). For example, FVIII can be coated directly or indirectly through a FVIII antibody to a Biacore™ chip, and varying concentrations of LRP can be passed over the chip to measure both on-rate and off-rate of the interaction (Bovenschen, et al., J. Biol. Chem. 278:9370-9377, 2003). The ratio of the two rates gives a measure of affinity. A two-fold, five-fold, ten-fold, or 30-fold decrease in affinity upon PEGylation would be desired.

Degradation of a FVIII by the protease APC can be measured by any of the methods known to those of skill in the art.

In one embodiment, the method comprises administering a biocompatible polymer which is covalently attached to the polypeptide at one or more of the FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118, and 2284. In another embodiment, the biocompatible polymer is covalently attached to the polypeptide at one or more of FVIII amino acid positions 377, 378, 468, 491, 504, 556, 1795, 1796, 1803, 1804, 1808, 1810, 1864, 1903, 1911, and 2284 and (1) the binding of the conjugate to low-density lipoprotein receptor related protein is less than the binding of the unconjugated polypeptide to the low-density lipoprotein receptor related protein; (2) the binding of the conjugate to low-density lipoprotein receptor is less than the binding of the unconjugated polypeptide to the low-density lipoprotein receptor; or (3) the binding of the conjugate to both low-density lipoprotein receptor related protein and low-density lipoprotein receptor is less than the binding of the unconjugated polypeptide to the low-density lipoprotein receptor related protein and the low-density lipoprotein receptor.

In a further embodiment, the method comprises administering a biocompatible polymer which is covalently attached to the polypeptide at one or more of FVIII amino acid positions 377, 378, 468, 491, 504, 556, and 711 and the binding of the conjugate to heparin sulfate proteoglycan is less than the binding of the unconjugated polypeptide to heparin sulfate proteoglycan. In a further embodiment, the biocompatible polymer is covalently attached to the polypeptide at one or more of the FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118, and 2284 and the conjugate has less binding to FVIII inhibitory antibodies than the unconjugated polypeptide. In a further embodiment, the biocompatible polymer is covalently attached to the polypeptide at one or more of the FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118, and 2284, for example, at one or more of positions 377, 378, 468, 491, 504, 556, and 711 and the conjugate has less degradation from a plasma protease capable of FVIII degradation than does the unconjugated polypeptide. The plasma protease may be activated protein C.

In a further embodiment, the method comprises administering a biocompatible polymer which is covalently attached to B-domain deleted FVIII at amino acid position 129, 491, 1804, and/or 1808. In a further embodiment, the biocompatible polymer is attached to the polypeptide at FVIII amino acid position 1804 and comprises polyethylene glycol. The one or more predefined sites for biocompatible polymer attachment may be controlled by site specific cysteine mutation.

One or more sites, for example, one or two, on the functional FVIII polypeptide may be the predefined sites for polymer attachment. In particular embodiments, the polypeptide is mono-PEGylated or diPEGylated.

The invention also relates to a method for the preparation of the conjugate comprising mutating a nucleotide sequence that encodes for the functional FVIII polypeptide to substitute a coding sequence for a cysteine residue at a pre-defined site; expressing the mutated nucleotide sequence to produce a cysteine enhanced mutein; purifying the mutein; reacting the mutein with the biocompatible polymer that has been activated to react with polypeptides at substantially only reduced cysteine residues such that the conjugate is formed; and purifying the conjugate. In another embodiment, the invention provides a method for site-directed PEGylation of a FVIII mutein comprising: (a) expressing a site-directed FVIII mutein wherein the mutein has a cysteine replacement for an amino acid residue on the exposed surface of the FVIII mutein and that cysteine is capped; (b) contacting the cysteine mutein with a reductant under conditions to mildly reduce the cysteine mutein and to release the cap; (c) removing the cap and the reductant from the cysteine mutein; and (d) at least about 5 minutes, at least 15 minutes, at least 30 minutes after the removal of the reductant, treating the cysteine mutein with PEG comprising a sulfhydryl coupling moiety under conditions such that PEGylated FVIII mutein is produced. The sulfhydryl coupling moiety of the PEG is selected from the group consisting of thiol, triflate, tresylate, aziridine, oxirane, S-pyridyl and maleimide moieties.

The invention also concerns pharmaceutical compositions for parenteral administration comprising therapeutically effective amounts of the conjugates of the invention and a pharmaceutically acceptable adjuvant. Pharmaceutically acceptable adjuvants are substances that may be added to the active ingredient to help formulate or stabilize the preparation and cause no significant adverse toxicological effects to the patient. Examples of such adjuvants are well known to those skilled in the art and include water, sugars such as maltose or sucrose, albumin, salts, etc. Other adjuvants are described, for example, in Remington's Pharmaceutical Sciences by E. W. Martin. Such compositions will contain an effective amount of the conjugate hereof together with a suitable amount of vehicle in order to prepare pharmaceutically acceptable compositions suitable for effective administration to the host. For example, the conjugate may be parenterally administered to subjects suffering from hemophilia A at a dosage that may vary with the severity of the bleeding episode. The average doses administered intravenously for hemophilia A are in the range of 40 units per kilogram for pre-operative indications, 15 to 20 units per kilogram for minor hemorrhaging, and 20 to 40 units per kilogram administered over an 8 hours period for a maintenance dose. For treatment of vWD, the dosage may be from 25-400 IU per kilogram. Other useful dosages for vWD are from 25-50, 25-100, 50-75, 50-100, 100-200, 150-200, 200-300, 250-300, 300-350, 300-400, 25-250, 100-400 and 200-400 IU/kg. Lower dosages are useful for prophylaxis and higher dosages are useful for the immune tolerance induction in patients having FVIII inhibitors.

In one embodiment, the inventive method involves replacing one or more surface BDD amino acids with a cysteine, producing the cysteine mutein in a mammalian expression system, reducing a cysteine which has been capped during expression by cysteine from growth media, removing the reductant to allow BDD disulfides to reform, and reacting with a cysteine-specific biocompatible polymer reagent, such as such as PEG-maleimide. Examples of such reagents are PEG-maleimide with PEG sizes such as 5, 22, or 43 kD available from Nektar Therapeutics of San Carlos, Calif. under Nektar catalog numbers 2D2M0H01 mPEG-MAL MW 5,000 Da, 2D2M0P01 mPEG-MAL MW 20 kD, 2D3X0P01 mPEG2-MAL MW 40 kD, respectively, or 12 or 33 kD available from NOF Corporation, Tokyo, Japan under NOF catalog number Sunbright ME-120MA and Sunbright ME-300MA, respectively. The PEGylated product is purified using ion-exchange chromatography to remove unreacted PEG and using size-exclusion chromatography to remove unreacted BDD. This method can be used to identify and selectively shield any unfavorable interactions with FVIII such as receptor-mediated clearance, inhibitory antibody binding, and degradation by proteolytic enzymes. We noted that the PEG reagent supplied by Nektar or NOF as 5 kD tested as 6 kD in our laboratory, and similarly the PEG reagent supplied as linear 20 kD tested as 22 kD, that supplied as 40 kD tested as 43 kD and that supplied as 60 kD tested as 64 kD in our laboratory. To avoid confusion, we use the molecular weight as tested in our laboratory in the discussion herein, except for the 5 kD PEG, which we report as 5 kD as the manufacturer identified it.

In addition to cysteine mutations at positions 491 and 1808 of BDD (disclosed above), positions 487, 496, 504, 468, 1810, 1812, 1813, 1815, 1795, 1796, 1803, and 1804 were mutated to cysteine to potentially allow blockage of LRP binding upon PEGylation. Also, positions 377, 378, and 556 were mutated to cysteine to allow blockage of both LRP and HSPG binding upon PEGylation. Positions 81, 129, 422, 523, 570, 1864, 1911, 2091, and 2284 were selected to be equally spaced on BDD so that site-directed PEGylation with large PEGs (>40 kD) at these positions together with PEGylation at the native glycosylation sites (41, 239, and 2118) and LRP binding sites should completely cover the surface of BDD and identify novel clearance mechanism for BDD.

In one embodiment, the cell culture medium contains cysteines that “cap” the cysteine residues on the mutein by forming disulfide bonds. In the preparation of the conjugate, the cysteine mutein produced in the recombinant system is capped with a cysteine from the medium and this cap is removed by mild reduction that releases the cap before adding the cysteine-specific polymer reagent. Other methods known in the art for site-specific mutation of FVIII may also be used, as would be apparent to one of skill in the art.

### Structure Activity Relationship Analysis of FVIII

FVIII and BDD FVIII are very large complex molecules with many different sites involved in biological reactions. Previous attempts to covalently modify them to improve pharmacokinetic properties had mixed results. That the molecules could be specifically mutated and then a polymer added in a site-specific manner was surprising. Furthermore, the results of improved pharmacokinetic properties and retained activity were surprising also, given the problems with past polymeric conjugates causing nonspecific addition and reduced activity.

In one embodiment, the invention concerns site-directed mutagenesis using cysteine-specific ligands such as PEG-maleimide. A non-mutated BDD does not have any available cysteines to react with a PEG-maleimide, so only the mutated cysteine position will be the site of PEGylation. More specifically, BDD FVIII has 19 cysteines, 16 of which form disulfides and the other 3 of which are free cysteines (McMullen, et al., Protein Sci. 4:740-746, 1995). The structural model of BDD suggests that all 3 free cysteines are buried (Stoliova-McPhie, et al., Blood 99:1215-1223, 2002). Because oxidized cysteines cannot be PEGylated by PEGmaleimides, the 16 cysteines that form disulfides in BDD cannot be PEGylated without being first reduced. Based on the structural models of BDD, the 3 free cysteines in BDD may not be PEGylated without first denaturing the protein to expose these cysteines to the PEG reagent. Thus, it does not appear feasible to achieve specific PEGylation of BDD by PEGylation at native cysteine residues without dramatically altering the BDD structure, which will most likely destroy its function.

The redox state of the 4 cysteines in the B domain of full-length FVIII is unknown. PEGylation of the 4 cysteines in the B domain may be possible if they do not form disulfides and are surface exposed. However, because full-length FVIII and BDD have a similar pharmacokinetic (PK) profile and similar half-lives in vivo (Gruppo, et al., Haemophilia 9:251-260, 2003), B domain PEGylation is unlikely to result in improved plasma half-life unless the PEG happens to also protect non-B domain regions.

To determine the predefined site on a polypeptide having FVIII activity for polymer attachment that will retain FVIII activity and improve pharmacokinetics, the following guidelines are presented based on BDD FVIII. Modifications should be targeted toward clearance, inactivation, and immunogenic mechanisms such as LRP, HSPG, APC, and inhibitory antibody binding sites. Stoilova-McPhie, et al., (Blood 99:1215-23, 2002) shows the structure of BDD. For example, to prolong half-life, a single PEG can be introduced at a specific site at or near LRP binding sites in A2 residues 484-509 and A3 residues 1811-1818. Introduction of the bulky PEG at these sites should disrupt FVIII's ability to bind LRP and reduce the clearance of FVIII from circulation. It is also believed that to prolong half-life without significantly affecting activity that a PEG can be introduced at residue 1648, which is at the junction of the B domain and the A3 domain in the full-length molecule and in the 14-amino acid liker I the BDD between the A2 and A3 domains.

Specificity of PEGylation can be achieved by engineering single cysteine residues into the A2 or A3 domains using recombinant DNA mutagenesis techniques followed by site-specific PEGylation of the introduced cysteine with a cysteine-specific PEG reagent such as PEG-maleimide. Another advantage of PEGylating at 484-509 and 1811-1818 is that these two epitopes represent two of the three major classes of inhibitory antigenic sites in patients. To achieve maximal effect of improved circulating half-life and reduction of immunogenic response, both A2 and A3 LRP binding sites can be PEGylated to yield a diPEGylated product. It should be noted that PEGylation within the 1811-1818 region may lead to significant loss of activity since this region is also involved in FIX binding. Site-directed PEGylation within 558-565 should abolish HSPG binding, but may also reduce activity as this region also binds to FIX.

Additional surface sites can be PEGylated to identify novel clearance mechanism of FVIII. PEGylation of the A2 domain may offer additional advantage in that the A2 domain dissociates from FVIII upon activation and is presumably removed from circulation faster than the rest of FVIII molecule because of its smaller size. PEGylated A2, on the other hand, may be big enough to escape kidney clearance and have a comparable plasma half-life to the rest of FVIII and thus can reconstitute the activated FVIII in vivo.

Identification of PEGylation Sites In A2 And A3 Regions. Five positions (Y487, L491, K496, L504 and Q468 corresponding to PEG1-5 positions) at or near the putative A2 LRP binding region were selected as examples for site-directed PEGylation based on the high surface exposure and outward direction of their Cα to Cβ trajectory. Furthermore, these residues are roughly equidistant from each other in the three-dimensional structure of the molecule, so that together they can represent this entire region. Eight positions (1808, 1810, 1812, 1813, 1815, 1795, 1796, 1803, 1804 corresponding to PEG6-14) at or near the putative A3 LRP binding region were selected as examples for site-directed PEGylation. PEG6 (K1808) is adjacent to 1811-1818 and the natural N-linked glycosylation site at 1810. PEGylation at position 1810 (PEG7) will replace the sugar with a PEG. Mutation at the PEG8 position T1812 will also abolish the glycosylation site. Although the PEGS position (K1813) was predicted to be pointing inward, it was selected in case the structure model is not correct. PEG10 (Y1815) is a bulky hydrophobic amino acid within the LRP binding loop, and may be a critical interacting residue since hydrophobic amino acids are typically found at the center of protein-protein interactions. Because the 1811-1818 region has been reported to be involved in both LRP and FIX binding, PEGylation within this loop was thought possibly to result in reduced activity. Thus, PEG11PEG14 (1795, 1796, 1803, 1804) were designed to be near the 1811-1818 loop but not within the loop so that one can dissociate LRP and FIX binding with different PEG sizes.

To block both LRP binding sites simultaneously, double PEGylation at, for example, the PEG2 and PEG6 position, can be generated.

Since the 558-565 region has been shown to bind to both HSPG and FIX, no sites were designed within this region. Instead, PEG15-PEG17 (377, 378, and 556) were designed in between the A2 LRP and HSPG binding regions so that an attached PEG may interfere both interactions and disrupt possible interactions between them. Additional sites that are surface exposed and outwardly pointing could also be selected within or near the LRP and HPSG binding regions. To identify novel clearance mechanisms, FVIII can be systematically PEGylated. In addition to PEG1-17, the three other natural glycosylation sites, namely, N41, N239, and N2118 corresponding to PEG18-20 can be used as tethering points for PEGylation since they should be surface exposed. Surface areas within a 20 angstrom radius from the Cβ atoms of PEG2, PEG6, and the four glycosylation sites were mapped onto the BDD model in addition to functional interaction sites for vWF, FIX, FX, phospholipid, and thrombin.

PEG21-29 corresponding to Y81, F129, K422, K523, K570, N1864, T1911, Q2091, and Q2284 were then selected based on their ability to cover nearly the entire remaining BDD surface with a 20 angstrom radius from each of their Cβ atoms. These positions were also selected because they are fully exposed, outwardly pointing, and far away from natural cysteines to minimize possible incorrect disulfide formation. The 20 angstrom radius is chosen because a large PEG, such as a 64 kD branched PEG, is expected to have the potential to cover a sphere with about a 20 angstrom radius. PEGylation of PEG21-29 together with PEG2 and PEG6 and glycosylation sites PEG18, 19, and 20 is likely to protect nearly the entire non-functional surface of FVIII.

PEGylation positions that lead to enhanced properties such as improved PK profile, greater stability, or reduced immunogenicity can be combined to generate multi-PEGylated product with maximally enhanced properties. PEG30 and PEG31 were designed by removing the exposed disulfides in A2 and A3 domain, respectively. PEG30, or C630A, should free up its disulfide partner C711 for PEGylation. Likewise, PEG31, C1899A should allow C1903 to be PEGylated.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Mutagenesis

Substrates for site-directed PEGylation of FVIII may be generated by introducing a cysteine codon at the site chosen for PEGylation. The Stratagene cQuickChange™ II site-directed mutagenesis kit was used to make all of the PEG mutants (Stratagene Corporation, La Jolla, Calif.). The cQuikChange™ site-directed mutagenesis method is performed using PfuTurbo® DNA polymerase and a temperature cycler. Two complimentary oligonucleotide primers, containing the desired mutation, are elongated using PfuTurbo®, which will not displace the primers. dsDNA containing the wildtype FVIII gene is used as a template. Following multiple elongation cycles, the product is digested with DpnI endonuclease, which is specific for methylated DNA. The newly synthesized DNA, containing the mutation, is not methylated, whereas the parental wild-type DNA is methylated. The digested DNA is then used to transform XL-1 Blue super-competent cells.

The mutagenesis reactions were performed in either pSK207+BDD C2.6 or pSK207+BDD. A description of the site-directed mutagenesis of FVIII purification of muteins, PEGylation, and activity measurements may be found in WO 2006/053299 which is incorporated herein by reference. A summary of the muteins is provided in Table 1.

TABLE 1

| Mutation | Mutein ID |
|---|---|
| Y487C | PEG1 |
| L491C | PEG2 |
| K496C | PEG3 |
| L504C | PEG4 |
| Q468C | PEG5 |
| K1808C | PEG6 |
| N1810C | PEG7 |
| T1812C | PEG8 |
| K1813C | PEG9 |
| Y1815C | PEG10 |
| D1795C | PEG11 |
| Q1796C | PEG12 |
| R1803C | PEG13 |
| K1804C | PEG14 |
| L491C/K1808C | PEG2 + 6 |
| L491C/K1804C | PEG2 + 14 |
| K377C | PEG15 |
| H378C | PEG16 |
| K556C | PEG17 |
| N41C | PEG18 |
| N239C | PEG19 |
| N2118C | PEG20 |
| Y81C | PEG21 |
| F129C | PEG22 |
| K422C | PEG23 |
| K523C | PEG24 |
| K570C | PEG25 |
| N1864C | PEG26 |
| T1911C | PEG27 |
| Q2091C | PEG28 |
| Q2284C | PEG29 |
| C630A | PEG30 |
| C1899A | PEG31 |

### Example 2

### vWF Binding ELISA

FVIII is allowed to bind to vWf in Severe Hemophilic Plasma in solution. The FVIII-vWf complex is then captured on a microtiter plate that has been coated with a vWf-specific monoclonal antibody. The FVIII bound to the vWf is detected with a FVIII polyclonal antibody and a horseradish peroxidase-anti-rabbit conjugate. The peroxidase-conjugated antibody complex produces a color reaction upon addition of the substrate. Sample concentrations are interpolated from a standard curve using four parameter fit model. FVIII binding results are reported in μg/mL. There was no significant impact on any of the activities upon PEGylation, which would be consistent with PEGylation at the B domain. Results may be found in Table 2.

TABLE 2

| | TAE | Coagulation Assay | | | Chromogenic Assay | | | vWF ELISA | | |
|---|---|---|---|---|---|---|---|---|---|---|
| Sample | ug/mL | IU/mL | IU/ug | % Start | IU/mL | IU/ug | % Start | ug/mL | vWF/TAE | % Start |
| KG-2 start | 1.31 | 4.8 | 3.6 | 100 | 5.60 | 4.3 | 100 | 0.42 | 0.32 | 100 |
| Reduced only | 0.93 | 3.1 | 3.4 | 93 | 4.08 | 4.4 | 103 | | | |
| KG-2-5 kD PEG | 0.71 | 2.5 | 3.5 | 96 | 3.09 | 4.3 | 102 | | | |
| KG-2-12 kD PEG | 0.59 | 2.3 | 3.9 | 107 | 2.99 | 5.0 | 118 | | | |
| KG-2-22 kD PEG | 0.63 | 2.5 | 3.9 | 108 | 3.06 | 4.8 | 113 | 0.19 | 0.30 | 94 |
| KG-2-30 kD PEG | 0.59 | 2.5 | 4.1 | 114 | 3.01 | 5.1 | 119 | 0.19 | 0.32 | 100 |
| KG-2-43 kD PEG | 0.52 | 2.4 | 4.6 | 128 | 2.86 | 5.5 | 129 | | | |

### Example 3

### Pharmacokinectic Activity

The PK of PEGylated FVIII and B domain-deleted FVIII (BDD-FVIII) was determined in FVIII knockout (KO) mice. The mice received an intravenous (i.v.) injection of 200 IU/kg BDD-FVIII, 108 IU/kg BDD-FVIII conjugated with 64 kD PEG at the cysteine mutation introduced at the amino acid position 1804 (64 kD PEG14), or 194 IU/kg of BDD-FVIII conjugated with 64 kD PEG at each of the cysteine mutations at positions 491 and 1804 (64 kD PEG2+14). Blood specimens were collected from treated mice (5 mice/treatment/time point) at 5 minutes, 4 hours, 8 hours, 16 hours, 24 hours, 32 hours, and 48 hours. Plasma FVIII activities were determined by Coatest assay. Terminal half-life was determined by non-compartment modeling of the activity vs time curve in WinNonLin. Whereas the t₁₁₂ for BDD-FVIII in FVIII KO mice is 6 hours, the t_{1/2} for FVIII conjugated with 64 kD PEG (64 kD PEG14) or 128 kD PEG (64 kD PEG2+14) is 12.43 hours and 12.75 hours, respectively. Therefore, the half-life of PEGylated FVIII was increased by about 2-fold in comparison to BDD-FVIII in FVIII KO mice.

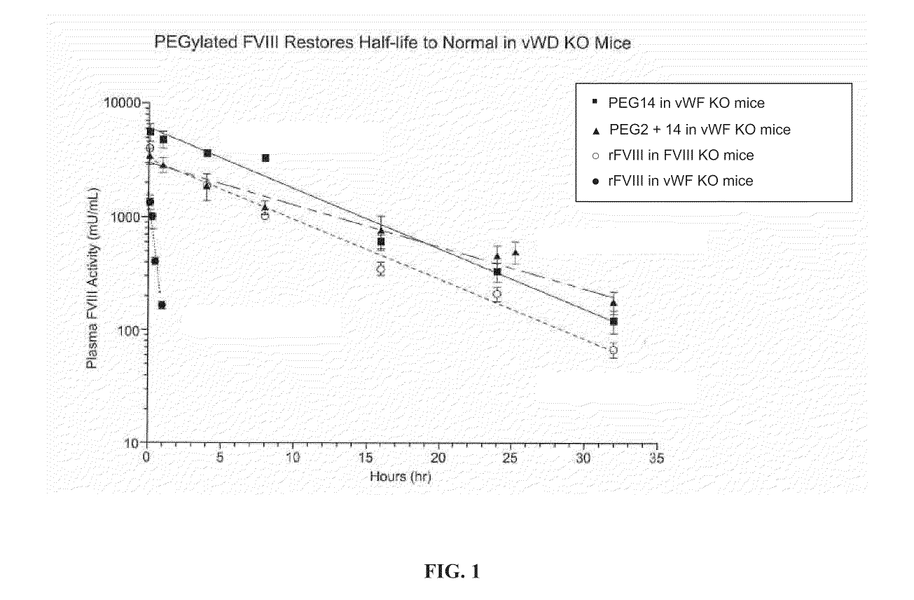

The absence of vWF in circulation eliminated the limit on the half-life extension of PEGylated FVIII, as demonstrated in vWF KO mice. Mice were dosed by i.v. administration of 200 IU/kg BDD-FVIII, 520 IU/kg of 64 kD PEG14, or 400 IU/kg of 64 kD PEG2+14. Blood specimens were collected at 5 minutes, 15 minutes, 30 minutes, 1 hour, 2 hours, 4 hours, 6 hours, and 8 hours from BDDFVIII treated mice, and at 5 minutes, 4 hours, 8 hours, 16 hours, 24 hours, 32 hours, and 48 hours from PEGylated FVIII treated mice (5 mice/treatment/time point). To eliminate the background activity from the endogenous murine FVIII, which is at about 2% of normal levels in the vWF KO mice, the plasma activity of infused human FVIII was measured by Capture Coatest. BDD-FVIII and PEGylated FVIII in plasma were first captured by mAb R8B12 (2 ug/mL) specific for the A3 domain of human FVIII, and then measured by the Coatest. In contrast to BDD-FVIII, which cleared rapidly without the protection from vWF, resulting in a t_{1/2} as short as 18 minutes, the t_{1/2} of 64 kD PEG14 and 64 kD PEG2+14 is 5.7 hours and 8.2 hours, respectively (FIG. 1). Thus, in contrast to the limited 2-fold increase in the t_{1/2} of PEG-FVIII compared to BDD-FVIII observed in the presence of vWF in the FVIII KO mice, the t_{1/2} of 64 kD PEG14 and 64 kD PEG2+14 are extended by 19- to 27-fold in the absence of vWF in the vWF KO mice. Furthermore, the increase in t_{1/2} of PEG-FVIII is proportional to the size of PEG.

All publications and patents mentioned in the above specification are incorporated herein by reference. Various modifications and variations of the described methods of the invention will be apparent to those skilled in the art without departing from the scope and spirit of the invention.

Although the invention has been described in connection with specific embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

We claim:

1. A method for treating von Willebrand Disease, comprising administering to a subject in need thereof a therapeutically effective amount of a conjugate that has FVIII procoagulant activity and that is capable of correcting human FVIII deficiencies, the conjugate comprising a functional FVIII polypeptide covalently attached at one or more predefined sites on the polypeptide to one or more biocompatible polymers, wherein the predefined site is a particular amino acid residue identified by numerical position in the amino acid sequence of the polypeptide and is not an N-terminal amine.

2. The method of claim 1, wherein the biocompatible polymer comprises polyethylene glycol.

3. The method of claim 2, wherein the polyethylene glycol comprises methoxypolyethylene glycol.

4. The method of claim 3, wherein the methoxypolyethylene glycol has a size range from 5 kD to 64 kD.

5. The method of claim 1, wherein the biocompatible polymer is covalently attached to the functional FVIII polypeptide at an amino acid residue in or near (a) a binding site for a FVIII clearance receptor, (b) a binding site for a protease capable of degradation of FVIII and/or (c) a binding site for FVIII inhibitory antibodies.

6. The method of claim 1, wherein the biocompatible polymer is covalently attached at the predefined site on the functional FVIII polypeptide such that binding of low-density lipoprotein receptor related protein to the polypeptide is less than to the polypeptide when it is not conjugated.

7. The method of claim 6, wherein the binding of low-density lipoprotein receptor related protein to the conjugate is less than one-half of the binding to the polypeptide when it is not conjugated.

8. The method of claim 1, wherein the biocompatible polymer is covalently attached at the predefined site on the functional FVIII polypeptide such that binding of heparan sulphate proteoglycans to the polypeptide is less than to the polypeptide when it is not conjugated.

9. The method of claim 8, wherein the binding of heparin sulphate proteoglycans to the conjugate is less than one-half of the binding to the polypeptide when it is not conjugated.

10. The method of claim 1, wherein the biocompatible polymer is covalently attached at the predefined site on the functional FVIII polypeptide such that binding of FVIII inhibitory antibodies to the polypeptide is less than to the polypeptide when it is not conjugated.

11. The method of claim 10, wherein the binding of FVIII inhibitory antibodies to the conjugate is less than one-half of the binding to the polypeptide when it is not conjugated.

12. The method of claim 1, wherein the biocompatible polymer is covalently attached at the predefined site on the functional FVIII polypeptide such that binding of low density lipoprotein receptor to the polypeptide is less than to the polypeptide when it is not conjugated.

13. The method of claim 12, wherein the binding of low density lipoprotein receptor to the conjugate is less than one-half of the binding to the polypeptide when it is not conjugated.

14. The method of claim 1, wherein the biocompatible polymer is covalently attached at the predefined site on the functional FVIII polypeptide such that a plasma protease degrades the polypeptide less than when the polypeptide is not conjugated.

15. The method of claim 14, wherein the degradation of the polypeptide by the plasma protease is less than one-half of the degradation of the polypeptide when it is not conjugated as measured under the same conditions over the same time period.

16. The method of claim 1, wherein the biocompatible polymer is covalently attached to the polypeptide at one of the FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118 and 2284 with reference to the mature, full-length human FVIII amino acid sequence of SEQ ID NO:1.

17. The method of claim 1, wherein the biocompatible polymer is covalently attached to the polypeptide at one or more of FVIII amino acid positions 377, 378, 468, 491, 504, 556, 1795, 1796, 1803, 1804, 1808, 1810, 1864, 1903, 1911 and 2284 with reference to the mature, full-length human FVIII amino acid sequence of SEQ ID NO:1 and further wherein (1) the binding of the conjugate to low-density lipoprotein receptor related protein is less than the binding of the unconjugated polypeptide to the low-density lipoprotein receptor related protein; (2) the binding of the conjugate to low-density lipoprotein receptor is less than the binding of the unconjugated polypeptide to the low-density lipoprotein receptor; or (3) the binding of the conjugate to both low-density lipoprotein receptor related protein and low-density lipoprotein receptor is less than the binding of the unconjugated polypeptide to the low-density lipoprotein receptor related protein and the low-density lipoprotein receptor.

18. The method of claim 1, wherein the biocompatible polymer is covalently attached to the polypeptide at one or more of FVIII amino acid positions 377, 378, 468, 491, 504, 556 and 711 with reference to the mature, full-length human FVIII amino acid sequence of SEQ ID NO:1 and further wherein the binding of the conjugate to heparin sulphate proteoglycan is less than the binding of the unconjugated polypeptide to heparin sulphate proteoglycan.

19. The method of claim 1, wherein the biocompatible polymer is covalently attached to the polypeptide at one or more of FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118 and 2284 with reference to the mature, full-length human FVIII amino acid sequence of SEQ ID NO:1 and the conjugate has less binding to FVIII inhibitory antibodies than the unconjugated polypeptide.

20. The method of claim 1, wherein the biocompatible polymer is covalently attached to the polypeptide at one or more of FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118 and 2284 with reference to the mature, full-length human FVIII amino acid sequence of SEQ ID NO:1 and the conjugate has less degradation from a plasma protease capable of FVIII degradation than does the unconjugated polypeptide.

21. The method of claim 20, wherein the plasma protease is activated protein C.

22. The method of claim 1, wherein the functional FVIII polypeptide is B-domain deleted FVIII.

23. The method of claim 22, wherein the biocompatible polymer is covalently attached to B-domain deleted FVIII at amino acid position 129, 491, 1804, and/or 1808 with reference to the mature, full-length human FVIII amino acid sequence of SEQ ID NO:1.

24. The method of claim 1, wherein the biocompatible polymer is attached to the polypeptide at FVIII amino acid position 1804 with reference to the mature, full-length human FVIII amino acid sequence of SEQ ID NO:1 and comprises polyethylene glycol.

25. The method of claim 1, wherein the one or more predefined sites for biocompatible polymer attachment is a cysteine residue.

26. The method of claim 1, wherein the von Willebrand Disease is characterized by a deficiency and/or abnormality of von Willebrand Factor.

27. The method of claim 1, wherein the von Willebrand Disease is Type N2.

28. The method of claim 1, wherein the von Willebrand Disease is Type 3.

29. A method of preparing a medicament for treating von Willebrand disease, comprising making a conjugate that has FVIII procoagulant activity and that is capable of correcting human FVIII deficiencies, the conjugate comprising a functional FVIII polypeptide covalently attached at one or more predefined sites on the polypeptide to one or more biocompatible polymers, wherein the predefined site is a particular amino acid residue identified by numerical position in the amino acid sequence of the polypeptide and is not an N-terminal amine.

30. A method for treating von Willebrand Disease, comprising administering to a subject in need thereof a therapeutically effective amount of a cysteine substituted variant of FVIII having FVIII procoagulant activity and capable of correcting human FVIII deficiencies, the variant characterized by having a cysteine residue substituted for an amino acid in the FVIII sequence, wherein said substitution causes a cysteine residue at an amino acid position where a cysteine residue is not present in FVIII with reference to the mature, full-length human FVIII amino acid sequence of SEQ ID NO:1, said cysteine added variant being further characterized by having a biocompatible polymer covalently attached to said substitute cysteine residue.

31. The method of claim 30, wherein the biocompatible polymer comprises polyethylene glycol.

32. A method for prophylactic treatment comprising administering to a subject in need thereof, prior to surgery, a therapeutically effective amount of a conjugate that has FVIII procoagulant activity and that is capable of correcting human FVIII deficiencies, the conjugate comprising a functional FVIII polypeptide covalently attached at one or more predefined sites on the polypeptide to one or more biocompatible polymers, wherein the predefined site is a particular amino acid residue identified by numerical position in the amino acid sequence of the polypeptide and is not an N-terminal amine, whereby episodic bleeding is attenuated.

33. The method of claim 32, wherein the subject has Type 3 vWD.

34. The method of claim 32, wherein the biocompatible polymer comprises polyethylene glycol.

35. The method of claim 32, wherein the one or more predefined sites for biocompatible polymer attachment is a cysteine residue.

36. A method for treatment of trauma, comprising administering to a trauma subject a therapeutically effective amount of a conjugate that has FVIII procoagulant activity and that is capable of correcting human FVIII deficiencies, the conjugate comprising a functional FVIII polypeptide covalently attached at one or more predefined sites on the polypeptide to one or more biocompatible polymers, wherein the predefined site is a particular amino acid residue identified by numerical position in the amino acid sequence of the polypeptide and is not an N-terminal amine, whereby episodic bleeding is attenuated.

37. The method of claim 36, wherein the subject has Type 3 vWD.

38. The method of claim 36, wherein the biocompatible polymer comprises polyethylene glycol.

39. The method of claim 36, wherein the one or more predefined sites for biocompatible polymer attachment is a cysteine residue.


SEQUENCE LISTING (4 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 4 aa, Artificial Sequence
  The first four amino acids for the B-domain of human Factor VIII sequence
SFSQ

SEQ ID NO: 3 - protein, 10 aa, Artificial Sequence
  The last ten amino acids for the B-domain of human Factor VIII sequence
NPPVLKRHQR

SEQ ID NO: 4 - protein, 1457 aa, Artificial Sequence
  Derived from human Factor VIII sequence
MQIELSTCFFLCLLRFCFSATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY